(19) United States
(12) Patent Application Publication
(10) Pub. No.: US 2019/0300932 A1
(43) Pub. Date: Oct. 3, 2019

(54) NANO-DETERMINISTIC LATERAL DISPLACEMENT ARRAY FOR NEXT-GENERATION SEQUENCING SAMPLE PREPARATION AND HIGH-SENSITIVITY DNA DETECTION

(71) Applicant: INTERNATIONAL BUSINESS MACHINES CORPORATION, Armonk, NY (US)

(72) Inventors: Stacey Gifford, Fairfield, CT (US); Benjamin Wunsch, Mt. Kisco, NY (US)

(21) Appl. No.: 15/942,726

(22) Filed: Apr. 2, 2018

(51) Int. Cl. C12Q 1/6806

(52) U.S. Cl. CPC C12Q 1/6806

(57) ABSTRACT

A method of purifying nucleic acid polymers for next generation sequencing includes loading a mixture of adapter ligated, fragmented nucleic acid polymers onto a nano-deterministic lateral displacement (nanoDLD) array. The mixture includes a first adapter ligated, fragmented nucleic acid polymer, a second adapter ligated, fragmented nucleic acid polymer, and a contaminant. The method further includes flowing the mixture through the nanoDLD array to separate the first adapter ligated, fragmented nucleic acid polymer from the second adapter ligated, fragmented nucleic acid polymer, and each of the first and second adapter ligated, fragmented nucleic acid polymers from the contaminant.

### BACKGROUND

The present invention generally relates to nucleic acid polymer sample preparation and detection. More specifically, the present invention relates to the application of a nano-deterministic lateral displacement (nanoDLD) array for next generation sequencing sample preparation and high sensitivity detection.

Next-generation sequencing (NGS), also known as high-throughput sequencing, refers to modern sequencing technologies that enable deoxyribonucleic acid (DNA) sequencing more quickly and easily than previously used sequencing techniques (e.g., Sanger methods). NGS has enabled the use of whole genome sequencing and single-cell sequencing in clinical applications, such as tumor characterization. Examples of NGS methods include, but are not limited to, SOLEXA sequencing methods available from Illumina, Inc., 454 sequencing methods available from Roche Sequencing, ION TORRENT methods available from ThermoFisher, and SOLiD sequencing methods available from Life Technologies, ThermoFisher.

### SUMMARY

Embodiments of the present invention are directed to a method for purifying nucleic acid polymers for next generation sequencing. A non-limiting example of the method includes loading a mixture of adapter ligated, fragmented nucleic acid polymers onto a nano-deterministic lateral displacement (nanoDLD) array. The mixture includes a first adapter ligated, fragmented nucleic acid polymer, a second adapter ligated, fragmented nucleic acid polymer, and a contaminant. The method further includes flowing the mixture through the nanoDLD array to separate the first adapter ligated, fragmented nucleic acid polymer from the second adapter ligated, fragmented nucleic acid polymer, and each of the first and second adapter ligated, fragmented nucleic acid polymers from the contaminant.

Embodiments of the present invention are also directed to method of detecting nucleic acid polymers for next generation sequencing. A non-limiting example of the method includes loading a mixture of adapter ligated, fluorescently tagged, fragmented nucleic acid polymers onto a nano-deterministic lateral displacement (nanoDLD) array. The mixture includes a first adapter ligated, fluorescently tagged, fragmented nucleic acid polymer and a second adapter ligated, fluorescently tagged, fragmented nucleic acid polymer. The method further includes flowing the mixture through the nanoDLD array to separate the first adapter ligated, fluorescently tagged, fragmented nucleic acid polymer from the second adapter ligated, fluorescently tagged, fragmented nucleic acid polymer. The method includes detecting fluorescence of the mixture with a fluorescence detector. The method also includes quantifying, based on detecting fluorescence, each of the first adapter ligated, fluorescently tagged, fragmented nucleic acid polymer and the second adapter ligated, fluorescently tagged, fragmented nucleic acid polymer.

Embodiments of the present invention are further directed to a method of concentrating samples for next generation sequencing. A non-limiting example of the method includes loading a mixture of adapter ligated, fragmented nucleic acid polymers onto a nano-deterministic lateral displacement (nanoDLD) array. The mixture includes a first adapter ligated, fragmented nucleic acid polymer, a second adapter, ligated fragmented nucleic acid polymer, and a contaminant. The method includes flowing the mixture through the nanoDLD array to separate the first adapter ligated, fragmented nucleic acid polymer from the second adapter ligated, fragmented nucleic acid polymer, and each of the first and second adapter ligated, fragmented nucleic acid polymers from the contaminant. A concentration of the mixture is increased after flowing through the nanoDLD array.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a flow diagram describing a method according to embodiments of the invention;

FIG. 2 depicts a flow diagram describing a method according to embodiments of the invention;

FIG. 3A depicts a nanoDLD array according to embodiments of the invention;

FIG. 3B depicts a nanoDLD array according to embodiments of the invention;

FIG. 3C depicts a nanoDLD array according to embodiments of the invention;

FIG. 3D depicts a machine layout of a machine including the nanoDLD array according to embodiments of the invention;

FIG. 4A depicts a nanoDLD array performing a method according to embodiments of the invention;

FIG. 4B depicts a nanoDLD array performing a method according to embodiments of the invention;

FIG. 5A depicts a fluorescence microscope image of a nanoDLD array according to embodiments of the invention;

FIG. 5B depicts a fluorescence microscope image of a nanoDLD array according to embodiments of the invention;

FIG. 5C depicts a fluorescence microscope image of a nanoDLD array according to embodiments of the invention;

FIG. 6 depicts a nanoDLD array for performing a method according to embodiments of the invention;

FIG. 7 depicts a schematic and fluorescence microscope image of a nanoDLD array performing a method according to embodiments of the invention; and

FIG. 8 depicts a schematic and fluorescence microscope image of a nanoDLD array performing a method according to embodiments of the invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the described embodiments, the various elements illustrated in the figures are provided with two or three digit reference numbers. With minor exceptions, the leftmost digit(s) of each reference number correspond to the figure in which its element is first illustrated.

### DETAILED DESCRIPTION

For the sake of brevity, conventional techniques related to use and fabrication of nanoDLD arrays (or nanodeterministic lateral displacement (nanoDLD) devices) may or may not be described in detail herein. Moreover, the various tasks and process steps described herein can be incorporated into a more comprehensive procedure or process having additional steps or functionality not described in detail herein. In particular, various steps in the use and fabrication of nanoDLD arrays are well known and so, in the interest of brevity, many conventional steps will only be mentioned briefly herein or will be omitted entirely without providing the well-known process details.

Turning now to an overview of technologies that are more specifically relevant to aspects of the invention, a limiting factor in the speed and accessibility of NGS is the time consuming steps needed for library preparation and validation. The general steps needed for NGS library preparation can include, for example, template fragmentation; reverse transcription (for RNA sequencing); end processing and adapter ligation; size selection and adapter dimer removal; amplification; and purification. Many of these individual steps also need a DNA purification method.

Adapter ligation includes the ligation of universal DNA adapters to the target sequence inserts, which allows for complete sequencing of all fragments. However, such ligation can also lead to a high background when adapter dimers form, which can overwhelm the actual sequencing step and thus decrease the number of measured meaningful sequence reads. Current methods to remove adapter dimers include, for example, gel purification and polyethylene glycol (PEG) based methods, which can suffer from low yield and low selectivity. A rapid method for high yield purification of specific DNA fragment sizes is needed for multiple steps in NGS library preparation.

In addition to purification methods, NGS sequencing also needs DNA detection methods. NGS sequencing includes amplification steps so that the library quality can be validated before time and resources are invested into sequencing. Current methods for validation include, for example, gel electrophoreses and bioanalyzer analysis, which can need a minimum of 1 nanogram or 0.5 ng/μL of DNA. These concentration levels are generally much higher than the starting template concentrations, particularly for methods such as single cell sequencing where essentially only one copy is available. Therefore, amplification is used to produce enough template to detect by standard methods and validate that the library is of sufficient quality for sequencing. A method that would allow for high-sensitivity or single molecule detection with small volumes of DNA would eliminate the need for amplification, which can introduce noise and error and increase the efficiency of NGS library preparation.

Turning now to an overview of the aspects of the invention, one or more embodiments of the invention address the above-described shortcomings of the prior art by providing methods that utilize a nanoscale deterministic lateral displacement (nanoDLD) array or nanopillar array to separate DNA fragments. According to one or more embodiments of the invention, DNA fragments as small as 100 base pairs can be separated based on size. Fragments above a critical size defined by the geometry of the nanopillar array will be bumped to the side of the array, while the smaller particles will flow through unperturbed in the zig-zag mode. The bump fraction can be collected, which provides a purified DNA sample of a specific length. The methods can be used for size selection and to remove impurities, such as adapter-dimers. According to one or more embodiments, DNA detection and size analysis can also be performed using fluorescent DNA labels at a single molecule level. The angle of migration in the array corresponds to the length of the DNA, which enables simultaneous counting and size measurement of low-yield samples and thus eliminates the need for amplification steps common to NGS library preparation workflows.

The above-described aspects of the invention address the shortcomings of the prior art by providing methods with advantages including high sensitivity, size-selectivity, yield, and efficiency. The methods enable single-particle detection and utilizes low sample volume for detection and preparation.

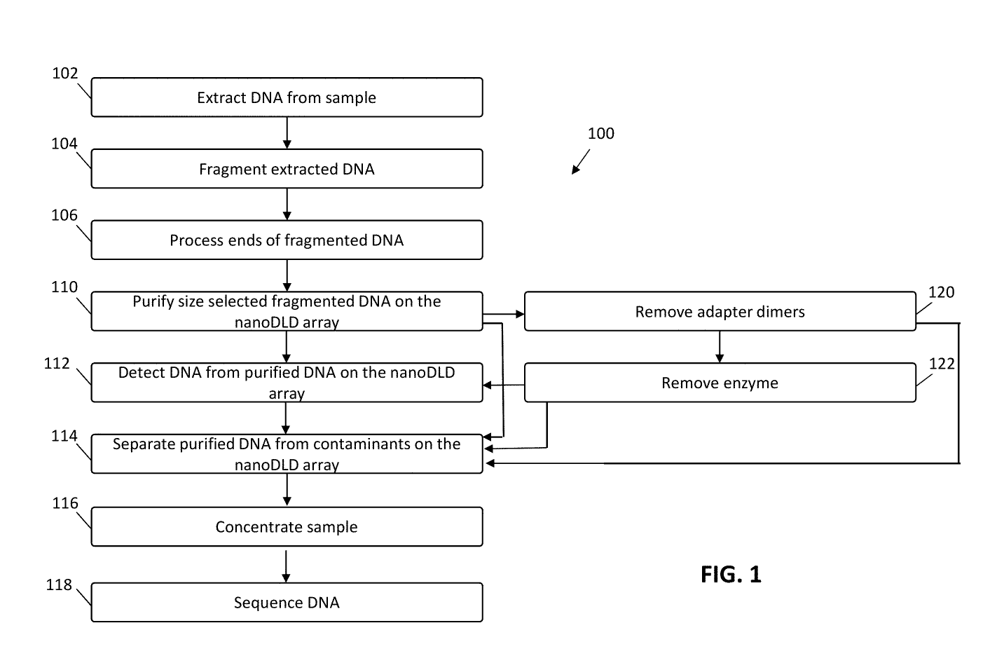

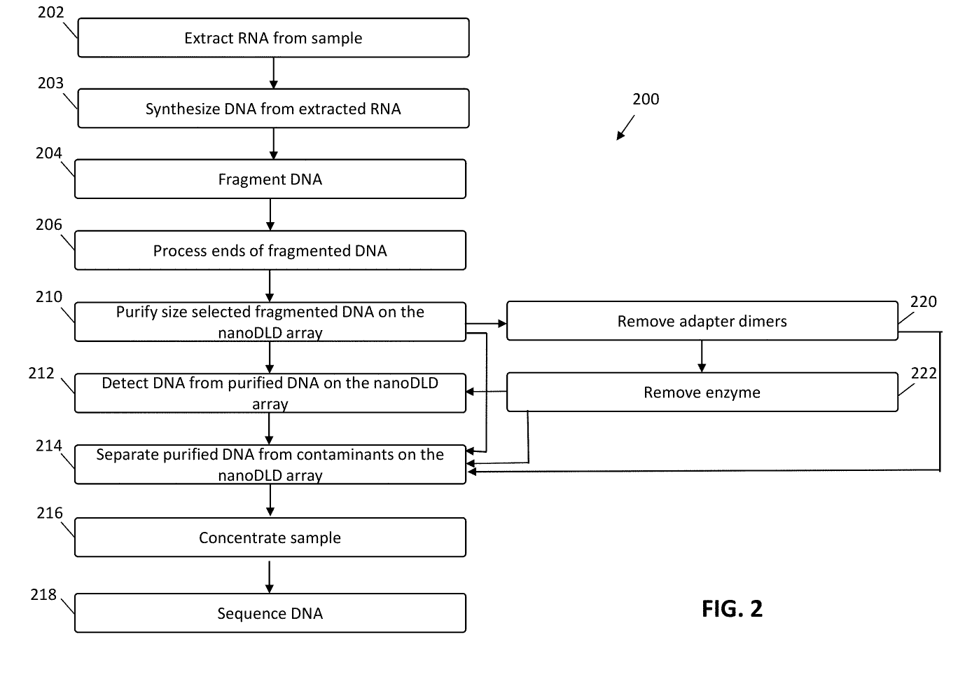

Turning now to a more detailed description of aspects of the present invention, FIGS. 1 and 2 depict flow diagrams describing methods 100 and 200 according to embodiments of the invention, which describe sample preparation for NGS sequencing. FIG. 1 describes a method directed to a starting sample of DNA, and FIG. 2 describes a method directed to a starting sample of RNA. Therefore, the methods described herein are generally directed to nucleic acid polymers (both DNA and RNA), and the below detailed discussion in the context of the method 100 of FIG. 1 also applies to the method 200 of FIG. 2.

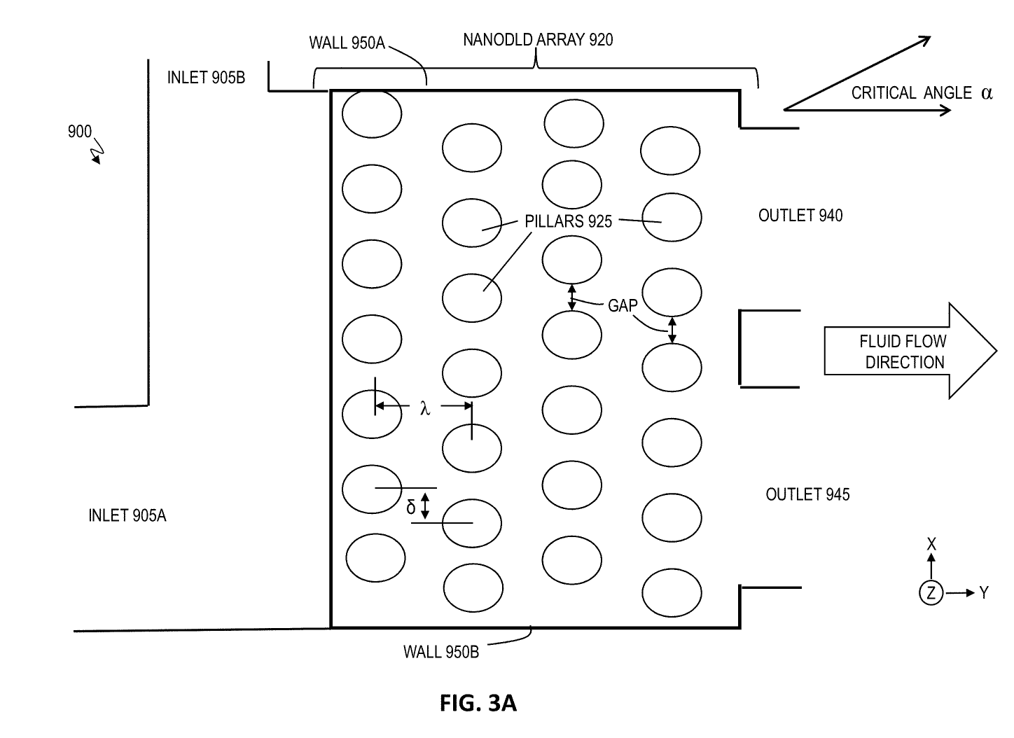

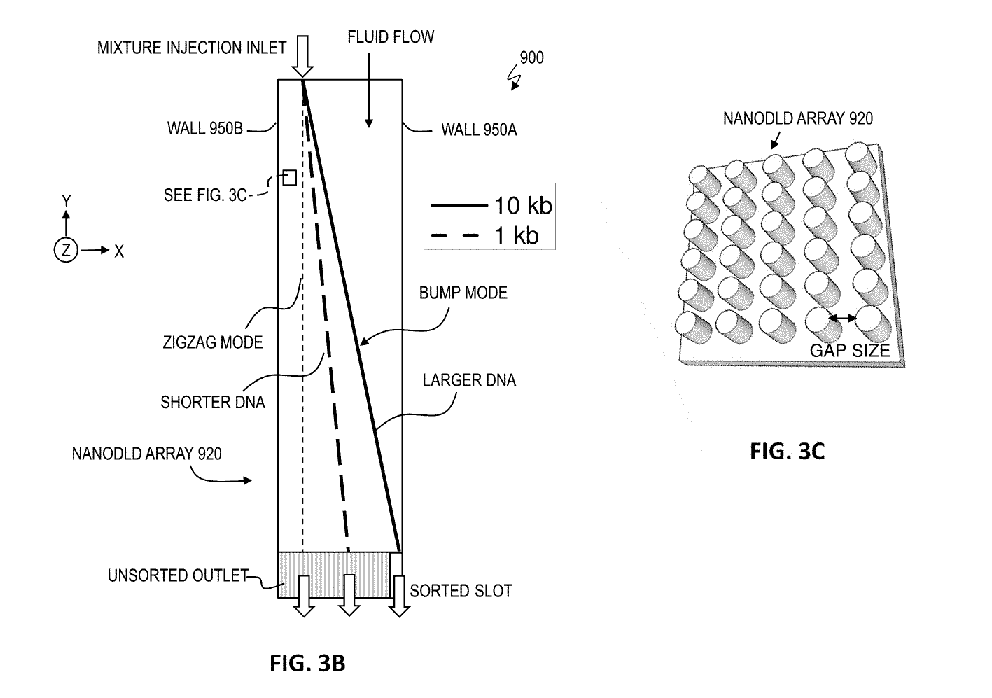

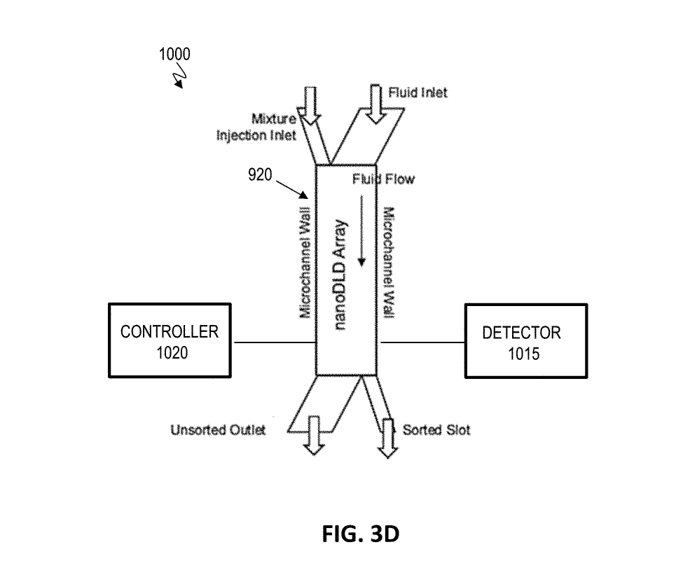

FIG. 1 depicts a flow diagram describing a method 100 according to embodiments of the invention. The method 100 utilizes a nanoDLD array 920 or nanopillar array, which is described in further detail in FIGS. 3A-3D below. The nanoDLD array 920 can be used in several areas of NGS sample preparation to increase efficiency. Methods steps in which the nanoDLD array 920 is used include, but are not limited to, areas needing size-specific DNA purification such as fragmentation (e.g., box 110 of method 100 and box 210 of method 200), high sensitivity detection (e.g., box 112 of method 100 and box 212 of method 200), and final library clean-up (e.g., box 114 of method 100 and box 214 of method 200).

The method 100 includes, at box 102, manually extracting DNA (a nucleic acid polymer) from a sample. The sample can be any type of cell, tissue, of organ having any origin, for example, eukaryotic or bacterial. According to one or more embodiments, the sample is blood or a cell. Physical and/or chemical methods can be used to extract the DNA from the sample. For example, the cells are lysed open to expose the cell membranes by, for example, by grinding or vortexing. Detergent is added to the lysed cells to disrupt the membranes, and cellular debris and proteins are removed by, for example, filtering and/or adding proteases. Alcohol can then be added to precipitate the DNA.

The method 100 includes, at box 104, fragmenting the extracted DNA (nucleic acid polymers) to form fragmented DNA (fragmented nucleic acid polymers). The fragmented nucleic acid polymer mixture includes fragments having a variety of lengths. The DNA can be fragmented by, for example, manually French pressing the extracted DNA samples, adding restriction enzymes to digest and cleave the DNA strands, sonicating the extracted DNA, or a combination thereof.

The method 100 includes, at box 106, processing ends of the fragmented DNA. Manually processing ends of the fragmented DNA includes, but is not limited to, ‘5 and 3’ end-repair, phosphorylation of the 5′ prime ends, A-tailing of the 3′ ends to facilitate ligation to sequencing adapters, and ligation of adapters. An adapter or linker is a short, chemically synthesized, single-stranded or double-stranded oligonucleotide that is be ligated to the ends of DNA or RNA. Adapter ligated fragmented DNA (nucleic acid polymers) are formed.

The method 100 includes, at box 110, purifying the size selected fragmented DNA on the nanoDLD array. The mixture of adapter ligated fragmented DNA is loaded onto and flowed through the nanoDLD array to separated adapter ligated fragmented DNA from contaminant(s). The mixture includes adapter ligated fragmented DNA having different lengths, as well as one or more contaminants. According to one or more embodiments, the mixture includes a first adapter ligated fragmented DNA strand and a second adapter ligated fragmented DNA strand, and the first and second strands are different lengths.

According to one or more embodiments, purifying the size selected fragmented DNA includes, at box 120, removing adapter dimers. Adapter dimers form as a result of self-ligation. The dimers can form clusters and consume valuable space without generating any useful data. According to other embodiments, purifying size selected DNA fragments, as shown at box 122, includes removing the enzyme used to fragment the DNA when an enzyme is used. Other impurities, such as un-fragmented DNA also can be removed. Removing impurities and undesired size-selected DNA eliminates unnecessary background noise.

FIG. 3A depicts a chip 900 (fluidic device) having a nanoDLD array 920, which can be used to purify the size selected fragmented DNA according to embodiments of the invention. FIG. 3B depicts a chip 900 (fluidic device) having a nanoDLD array 920, which can be used to purify the size selected fragmented DNA according to embodiments of the invention. FIG. 3C is an enlarged view illustrating that the nanoDLD array 920 is utilized for deterministic lateral displacement according to one or more embodiments.

The nanoDLD array 920 includes an array of pillars 925. The chip 900 has at least one inlet 905A to receive fluid containing fragmented DNA and contaminants to be separated. The inlet 905A can be an opening or hole in the walls around the nanoDLD array 920 or can span the width of the nanoDLD array 920 through which fluid (e.g., water, electrolyte solutions, organic solvents, etc.) and the mixture of DNA with contaminants can flow. In one implementation, there can be two or more inlets 905A and 905B. In this case, the inlet 905A receives input of the mixture to be sorted, and the mixture can be in a fluid (such as an electrolyte solution). The inlet 905B can be utilized to input a fluid, such as a buffer, not containing the mixture of the particles.

DNA fragments having a size greater than the critical dimension are bumped (i.e., bump mode, see FIG. 3B) through the nanoDLD array 920 in the direction of the critical angle a, and fragments larger than the critical dimension are laterally displaced in the x-axis and collected at outlet 940. The critical dimension is the size (e.g., diameter or length) of a contaminant (e.g., adapter dimers or enzyme) and/or persistence length of fragmented DNA that is too large to zigzag through the nanoDLD pillar array 920. On the other hand, contaminants or fragmented DNA having a size smaller than the critical dimension zigzag (i.e., zigzag mode, see also FIG. 3B) through the nanoDLD array 920 in the direction of fluid flow, and these smaller particles are collected (with very little lateral displacement and/or relatively no lateral displacement in the x-axis) at the outlet 945. The particles having the size smaller than the critical dimension follow the direction of the fluid flow, and are sorted through the outlet 945. The outlets 940 and 945 can be openings through which the sorted particles can flow and be collected in bins after sorting. It is appreciated that although only two outlets 940 and 945 are depicted, there can more than two outlets to provide more sorted DNA fragments. For example, there can be 3, 4, 5 or more outlets for sorting different sized fragmented DNA and contaminants.

The nanoDLD array 920 is a deterministic lateral displacement (DLD) array with predefined array parameters. The pillars 925 are periodically arranged with spacing λ, and each downstream row (rows run in the x-axis) is offset laterally from the previous row by the amount δ breaking the symmetry of the array. This array axis forms an angle α=tan₋₁ (δ/λ)=tan⁻¹(ε) with respect to the channel walls 950A, 950B and therefore the direction of fluid flow. Because of the array asymmetry, fluid flow in the gaps (Gaps) between the posts/pillars G is partitioned into 1/ε slots. Each of these slots repeats every 1/ε rows so the flow through the array is on average straight. Fragments transiting the gap G near a post can be displaced into an adjacent streamline if the particle's radius, or effective radius in the case of tumbling oblong objects such as rods with a defined length, is larger than the slot width in the gap. Therefore, larger fragments are deterministically displaced at each post and migrate at an angle α (critical angle) to the flow. Smaller fragments simply follow the streamline paths and flow through the array in the direction of fluid flow.

During operation, fragments greater than the predefined critical size are displaced laterally (in the x-axis) at each row by a pillar 925 and follow a deterministic path through the array in the so-called “bumping” or “bump” mode (see also FIG. 3B). The trajectory of bumping particles follows the array axis angle α. Particles smaller than the critical size follow the flow streamlines, weaving through the post array in a periodic “zigzag” mode (see also FIG. 3B). Therefore, array elements can be tailored to direct specific particle sizes at an angle to the flow by building arrays with design parameters shown in FIG.3A, which include obstacle size/length, spacing between the posts/pillars G, and post/pillar pitch λ. As noted above, asymmetry is determined by the magnitude of the row-to-row shift δ and is characterized by the slope ε=δ/λ, then leading to the final array angle being α=tan⁻¹(ε). For a given array angle, the critical particle size for the bumping mode is determined by the ratio between the particle diameter and the pillar spacing and/or gap.

It should be appreciated that the array elements and any ancillary microfluidic channels and reservoirs can be fabricated in silicon wafers by using standard microfabrication techniques including photolithography and etching. Arrays can also be molded in polydimethylsiloxane (PDMS) by using similarly crafted silicon. For the silicon etch, an optimized deep reactive ion etch (DRIE) can be used to maintain smooth, vertical side walls, ensuring uniform top-to-bottom spacing between posts/pillars. Embodiments are designed to create manufacturable silicon pillar arrays with uniform gaps between the pillars (also referred to as posts) with dimensions in the sub-100 nanometer (nm) regime. The pillar arrays can be designed with an oxide coating, such as a SiO₂ coating, which can be used to “heal” variation in the gap size along the entire axis of the pillars.

FIG. 3D depicts a machine layout of a machine 1000 that integrates a nanoDLD chip according to one or more embodiments. The machine 1000 can be used to perform any of the methods described herein that utilize a nanoDLD chip and/or nanoDLD array for methods of purifying fragmented DNA, detecting labeled fragmented DNA and/or concentrating DNA.

The machine 1000 can include a housing or encasing (not shown) which integrates a nanoDLD chip and any fluidic networks and injection ports required for transporting fluid samples into and off of the nanoDLD chip, as well as injecting/extracting fluid from the housing. The nanoDLD array 920 includes a mixture injection port for injecting the samples. A syringe pump can be used to inject the samples and fluids, which can be controlled by the controller 1020. However, the samples also can be manually injected. The nanoDLD array 920 optionally includes another fluid inlet for injecting fluids. A detector 1015 can be attached or mounted to the nanoDLD array 920 or can be a separate portable unit. The detector 1015 can be a full-sized fluorescence microscope for example. In one implementation, the fragmented DNA (and flow) can be detected by the detector 1015 through fluorescence microscopy of fluorophore labeled DNA molecules. The detector's output of the detector 1015 can be fed into the controller 1020. In a general operation, a complex mixture of DNA fragments and contaminants is fed into the nanoDLD array 920 through the injection inlet. The controller 1020 can control the sample injection, flow, and/or detector, for example. The controller 1020 can include a processor that is communicatively connected to an input device, a network, a memory, and a display. In some embodiments, the controller 1020 can include a personal computer, smart phone or tablet device communicatively connected to the fabrication machine 1000.

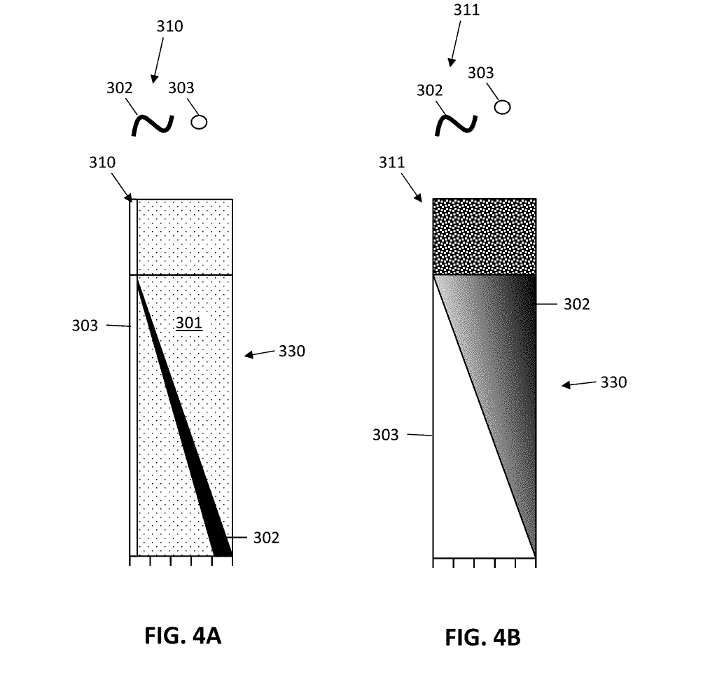

FIGS. 4A and 4B depict methods for purifying size selected fragmented DNA according to embodiments of the invention. FIGS. 4A and 4B show different methods of loading the samples into the nanoDLD array, both of which are pressure-driven systems. DNA purification is achieved by loading a sample containing the desired target DNA fragments 302 as well as any contaminants 303 that need to be removed, including but not limited to, adapter dimers, smaller DNA or RNA fragments, and enzymes into the nanoDLD array 330. The nanoDLD array 330 performs size-based separation based on the size of the gap between the pillars. The nanoDLD array 330 can separate fragments down to 100 base pair (bp) dsDNA. Flowing a mixture through the nanoDLD array separates at least a first adapter ligated fragmented nucleic acid polymer (e.g., DNA fragment) from a second fragmented nucleic acid polymer (e.g., DNA fragment), and each of the fragments from a contaminant, based on size.

The gap size between the pillars dictates a critical size, or DNA length for bumping. Particles in the nanoDLD array 330 below the critical size (contaminants 303) move through the array unaffected in zig-zag mode. Particles that are above the critical size (target DNA fragments) are displaced by the array to the side wall in bump mode. For the size selection to occur, the contaminants must be smaller than the target DNA fragment. The gap size between the pillars is selected based on the length of the target DNA 302 such that the target DNA 302 will fully bump and separate, while smaller contaminants 303 remain in zig-zag mode. Particles near the critical size exhibit partial bump mode and travel through the array at an angle of displacement concomitant with its size.

FIG. 4A shows a focused injection system, achieved by a condenser focuser or hydrodynamic focuser that condenses the sample to the left side of the channel as it enters the nanoDLD array 330. All contaminants 303 remain in zig-zag mode while the target DNA 302 particle is bumped to the far right wall of the channel and collected as a pure, concentrated sample. This sample loading method is selected when a high purity sample is desired or when a new buffer 301 is required.

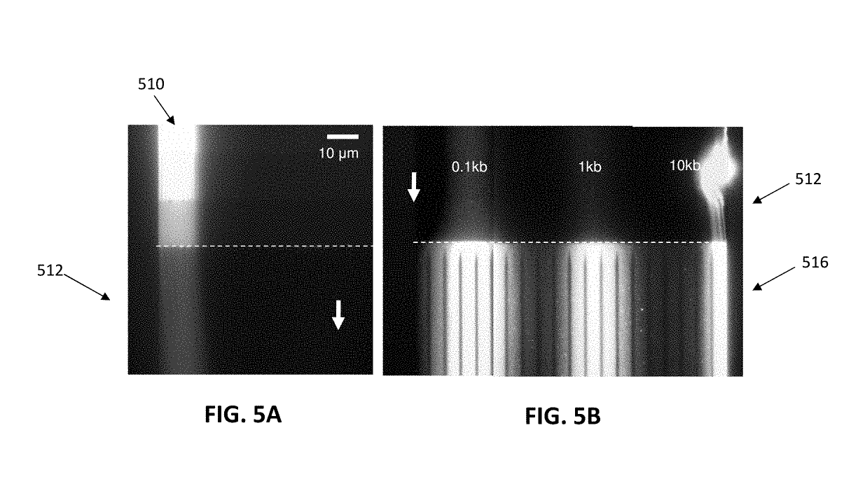

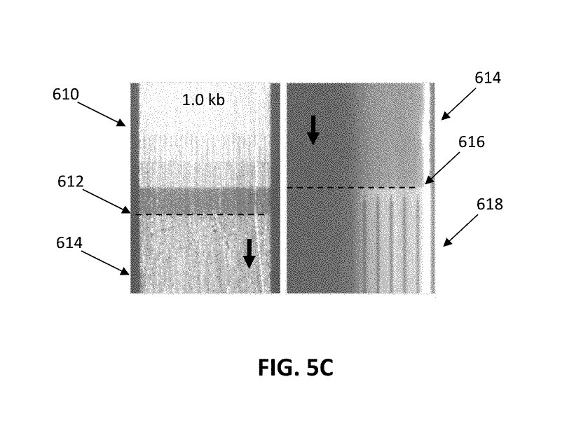

FIGS. 5A and 5B show a fluorescence microscope image of an example of a hydrodynamically-focused injection system that separates three DNA fragment sizes. FIG. 5A shows the inlet 510 where the sample that includes a combination of 0.1 kb, 1 kb, and 10 kb sized DNA fragments is injected into the nanoDLD array 512. The direction of sample/fluid flow is indicated by the arrow. FIG. 5B shows the outlet 516 from the nanoDLD array 512, with the direction of sample/fluid flow indicated by the arrow. As shown, the 0.1 kb, 1 kb, and 10 kb DNA fragments are separated as they exit from the nanoDLD array 512.

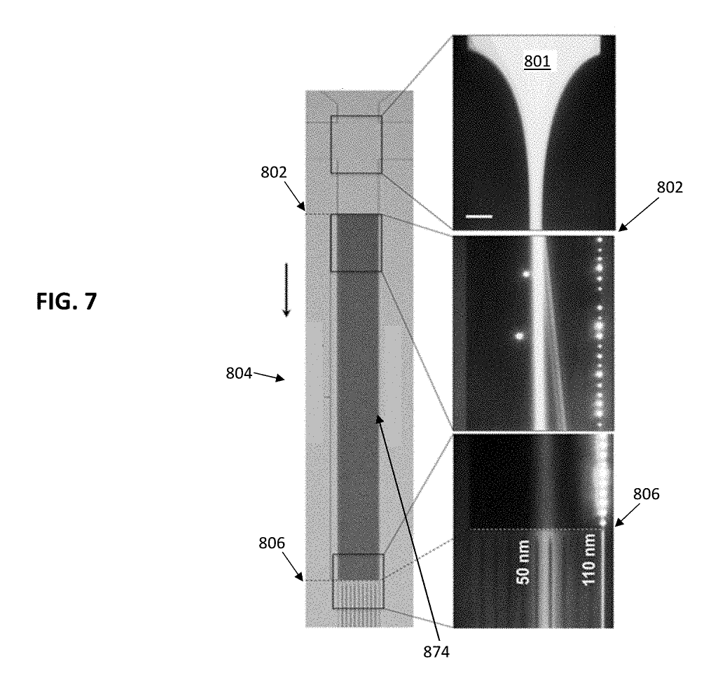

FIG. 7 depicts a focused sample injection device into a nanoDLD array according to embodiments of the invention. The samples are labeled with a fluorescent dye. In the exemplary device, the sample 801 is injected as a focused stream through the inlet 802 into the nanoDLD array 804. The collection wall 874 of the device is on the right side of the nanoDLD array 804. Although samples are collected at the outlet 806, as they flow through the nanoDLD array, some samples can hit the collection wall 874, which stops their lateral movement across the array. Flow then pushes the samples out through the bottom of the outlet 806. The collection wall 874 is where the samples concentrate before they flow out the outlet 806. The fragments are separated based on size as they exit through the outlet 806. Fragments of 50 nm and 100 nm lengths were separated from one another.

FIG. 4B shows a fluorescence image of a full-width injection system, where the sample 311 that includes the target DNA 302 and contaminants 303 enters across the entire width of the nanoDLD array 330. The smaller contaminants 303 remain in zigzag mode and enter out the full width of the array outlet. The larger target DNA 302 is bumped and concentrated to the right side of the array. In this example, one-fifth the final volume is collected as the bump fraction, but this fraction volume could be increased or decreased as necessary in order to alter the signal-to-noise ratio of the target DNA to the background contamination. Selecting a smaller collection volume for the bump fraction will also increase the signal-to-noise ratio. This method is selected when a highly pure sample is less critical and concentration is essential as no additional buffer is introduced into the system (compared to the method shown in FIG. 4A which introduces a buffer).

FIG. 5C shows a fluorescence microscope image of an example of a full-width injection system that concentrates a single DNA fragment size. On the left side of FIG. 5C, the sample 610 includes a 1.0 kb DNA fragment which is injected through the inlet 612 into the nanoDLD array 614. The direction of fluid flow is shown by the arrow. On the right side of FIG. 5C, the sample flows through the nanoDLD array 614 and exits the outlet 616 as a concentrated or enriched sample 618.

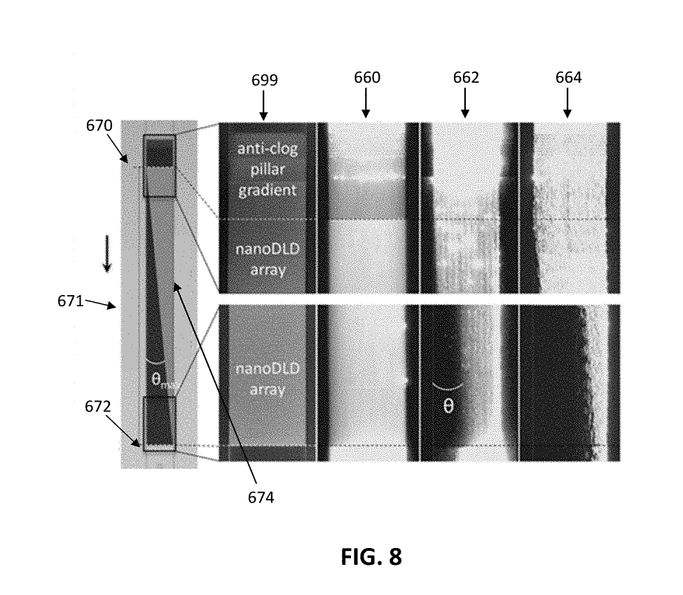

FIG. 8 depicts a full width sample injection device into a nanoDLD array according to embodiments of the invention. Three different sized particles are shown flowing through the same gap sized array. Each sample is injected across the full width of the array. Panel 699 shows the general layout of the array. The sample is injected at full width across the device through the inlet 670 into the nanoDLD array 671. The collection wall 674 of the device is on the right side of the nanoDLD array 671. The collection wall 674 is where the larger samples concentrate before they flow out the outlet 672. Depending on the size of the fragments in the sample, the samples will move through the nanoDLD array 671 in a different fashion. Panel 660 shows the smallest sample size, which moves through the nanoDLD array in zig-zag mode. Panel 664 shows the largest particle size, which moves through the nanoDLD array in a full bump mode. Panal 662 shows a particle that is near the critical diameter, which bumps through the nanoDLD array in a partial bump mode. The angle of the particle trajectory, as shown, correlates with its size. As the size of the particles increase, a shift in the angle of migration occurs until reaching a full bump mode.

Referring again to FIG. 1, after purifying the size selected DNA on the nanoDLD array, the method 100 includes either, as shown at box 112, detecting DNA from the purified DNA on the nanoDLD array or, as shown at box 114, separating purified DNA from contaminants on the nanoDLD array. Detecting DNA using the nanoDLD array replaces tradition PCR amplification that is generally needed for NGS sample preparation.

DNA detection can be achieved using the nanoDLD array by using a fluorescent dye that binds specifically to DNA or RNA as a tag. Examples of fluorescent dyes that can be used to tag the DNA or RNA include, but are not limited to, dimeric cyanine nucleic acid stains (e.g., YOYO-1 dyes), molecular beacon probes, asymmetrical cyanine dyes (e.g., SYBR Green I dyes), or a combination thereof. DNA samples to be tested are combined with a fluorescent dye and loaded on the nanoDLD array in a focused injection configuration, as shown in FIG. 4A for example. Fluorescence imaging of the array will be used to detect single DNA molecules as they traverse through the array, allowing for single molecule sensitivity. The angles at which the DNA particles migrate through the array correspond to their length, allowing for simultaneous size analysis. The technique offers a high-sensitivity replacement for current detection technique such as gel electrophoresis or bioanalyzer.

According to one or more embodiments, a mixture is flowed through the nanoDLD array to separate fluorescently tagged fragments from one another. At least a first adapter ligated, fluorescently tagged, fragmented nucleic acid polymer is separated from a second adapter ligated, fluorescently tagged, fragmented nucleic acid polymer. A detector is used to detect fluorescence of each of the tagged fragments, which are then quantified.

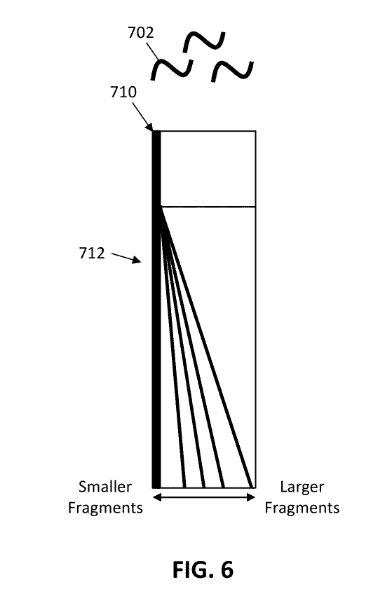

FIG. 6 depicts a method for detection and size analysis according to embodiments of the invention. A fluorescent dye-labeled sample 702 with different DNA or RNA fragments lengths is loaded by a focused injection system 710 into the nanoDLD array 712. The DNA or RNA fragments are separated according to length, with the larger fragments moving towards the right side of the nanoDLD array 712 and the smaller fragments flowing straight down. The angles at which the DNA or RNA fragments migrate through the array correspond to their length. After being separated, the number of particles of each length can be quantified.

The examples shown in FIGS. 5A and 5B discussed above demonstrate separation of three DNA fragment sizes that were labeled with a fluorescent dye. The tagged 0.1 kb, 1 kb, and 10 kb DNA fragments are separated as they exit from the nanoDLD array 512.

Referring again to FIG. 1, the method 100 includes, a box 116, optionally concentrating the sample. The sample is concentrated by loading the sample again into the nanoDLD array, as described above for the purification at box 110. Whether the sample needs to be concentrated depends on the intended end use of the sample and the initial concentration. The sample is loaded onto the nanoDLD array, and any remaining contaminants are separated from the DNA or RNA fragments.

A full width injection configuration can be used to increase concentration of the sample when needed. FIG. 4B described above provides an example of such a system where the smaller contaminants 303 remain in zigzag mode and exit out the full width of the array outlet, and the larger target DNA 302 is bumped and concentrated to the right side of the array. This method is selected when a highly pure sample is less critical and concentration is essential as no additional buffer is introduced into the system.

FIG. 5C described above provides another example of a full-width injection system that concentrates a single DNA fragment size. On the left side of FIG. 5C, the sample 610 includes a 1.0 kb DNA fragment which is injected through the inlet 612 into the nanoDLD array 614. On the right side of FIG. 5C, the sample flows through the nanoDLD array 614 and exits the outlet 616 as a concentrated or enriched sample 618.

Referring again to FIG. 1, the method 100 includes, at box 118, sequencing the DNA. The DNA fragments can be sequenced by PCR, for example.

FIG. 2 depicts a flow diagram describing a method 200 according to embodiments of the invention. The method 200 utilizes a nanoDLD array. The above discussion of the method 100 described in FIG. 1 directed to DNA applies to the method 200 described in FIG. 2 directed to RNA.

The method 200 includes, at box 202, extracting RNA from a sample. The method includes, at box 203, synthesizing double stranded DNA (complementary DNA) from the single stranded RNA. DNA is synthesized from a single stranded RNA (e.g., messenger RNA (mRNA) or microRNA) template in a reaction catalyzed by the enzyme reverse transcriptase. The method 200 includes, at box 204, fragmenting the DNA. The method 200 includes, at box 206, processing ends of the fragmented DNA. The method 200 includes, at box 210, purifying the size selected fragmented DNA on the nanoDLD array. According to one or more embodiments, purifying the size selected fragmented DNA includes, at box 220, removing adapter dimers. According to other embodiments, purifying size selected DNA fragments, as shown at box 222, includes removing the enzyme used to fragment the DNA when an enzyme is used.

After purifying the size selected DNA on the nanoDLD array, the method 200 includes either, as shown at box 212, detecting DNA from the purified DNA on the nanoDLD array or, as shown at box 214, separating purified DNA from contaminants on the nanoDLD array. The method 200 includes, a box 216, optionally concentrating the sample. The sample is concentrated by loading the sample again into the nanoDLD array, as described above for the purification at box 210. The method 200 includes, at box 218, sequencing the DNA.

Various embodiments of the present invention are described herein with reference to the related drawings. Alternative embodiments can be devised without departing from the scope of this invention. Although various connections and positional relationships (e.g., over, below, adjacent, etc.) are set forth between elements in the following description and in the drawings, persons skilled in the art will recognize that many of the positional relationships described herein are orientation-independent when the described functionality is maintained even though the orientation is changed. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. As an example of an indirect positional relationship, references in the present description to forming layer “A” over layer “B” include situations in which one or more intermediate layers (e.g., layer “C”) is between layer “A” and layer “B” as long as the relevant characteristics and functionalities of layer “A” and layer “B” are not substantially changed by the intermediate layer(s).

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include an indirect “connection” and a direct “connection.”

References in the specification to “one embodiment,” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper,” “lower,” “right,” “left,” “vertical,” “horizontal,” “top,” “bottom,” and derivatives thereof shall relate to the described structures and methods, as oriented in the drawing figures. The terms “overlying,” “atop,” “on top,” “positioned on” or “positioned atop” mean that a first element, such as a first structure, is present on a second element, such as a second structure, wherein intervening elements such as an interface structure can be present between the first element and the second element. The term “direct contact” means that a first element, such as a first structure, and a second element, such as a second structure, are connected without any intermediary conducting, insulating or semiconductor layers at the interface of the two elements.

The phrase “selective to,” such as, for example, “a first element selective to a second element,” means that the first element can be etched and the second element can act as an etch stop.

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

The flowchart and block diagrams in the Figures illustrate possible implementations of fabrication and/or operation methods according to various embodiments of the present invention. Various functions/operations of the method are represented in the flow diagram by blocks. In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A method of purifying nucleic acid polymers for next generation sequencing, the method comprising:
loading a mixture of adapter ligated, fragmented nucleic acid polymers onto a nano-deterministic lateral displacement (nanoDLD) array, the mixture comprising a first adapter ligated, fragmented nucleic acid polymer, a second adapter ligated, fragmented nucleic acid polymer, and a contaminant; and
flowing the mixture through the nanoDLD array to separate the first adapter ligated, fragmented nucleic acid polymer from the second adapter ligated, fragmented nucleic acid polymer, and each of the first and second adapter ligated, fragmented nucleic acid polymers from the contaminant.

2. The method of claim 1, wherein the first and second adapter ligated, fragmented nucleic acid polymers are fragmented deoxyribonucleic acid (DNA) polymers.

3. The method of claim 1 further comprising extracting nucleic acid polymers from a sample and subsequently fragmenting the extracted nucleic acid polymers prior to loading the mixture onto the nanoDLD array.

4. The method of claim 3, wherein the nucleic acid polymers extracted from the sample are RNA polymers, and the method further comprises synthesizing complementary DNA polymers from the RNA polymers.

5. The method of claim 1, wherein the contaminant comprises an enzyme used to fragment nucleic acid polymers, an adapter dimer, or a combination thereof.

6. The method of claim 1, wherein loading the mixture onto the nanoDLD array comprises using a focused injection configuration.

7. The method of claim 1, wherein loading the mixture onto the nanoDLD array comprises using a full width injection configuration.

8. A method of detecting nucleic acid polymers for next generation sequencing, the method comprising:
loading a mixture of adapter ligated, fluorescently tagged, fragmented nucleic acid polymers onto a nano-deterministic lateral displacement (nanoDLD) array, the mixture comprising a first adapter ligated, fluorescently tagged, fragmented nucleic acid polymer and a second adapter ligated, fluorescently tagged, fragmented nucleic acid polymer;
flowing the mixture through the nanoDLD array to separate the first adapter ligated, fluorescently tagged, fragmented nucleic acid polymer from the second adapter ligated, fluorescently tagged, fragmented nucleic acid polymer;
detecting fluorescence of the mixture with a fluorescence detector; and
quantifying, based on detecting fluorescence, each of the first adapter ligated, fluorescently tagged, fragmented nucleic acid polymer and the second adapter ligated, fluorescently tagged, fragmented nucleic acid polymer.

9. The method of claim 8, wherein the mixture comprises fragmented deoxyribonucleic acid (DNA) polymers.

10. The method of claim 8, wherein loading the mixture on the nanoDLD array comprises using a focused injection configuration.

11. The method of claim 8, wherein single molecules are detected by detecting fluorescence.

12. The method of claim 8 further comprising, subsequent to quantifying, separating the first and second adapter ligated, fluorescently tagged, fragmented nucleic acid polymers from contaminants on the nanoDLD array.

13. The method of claim 12, wherein the contaminants include adapter dimers, enzyme, or a combination thereof.

14. The method of claim 8, wherein the nanoDLD array comprises a first row and a second row of nanopillars, and the first row is laterally offset from the second row.

15. A method of concentrating samples for next generation sequencing, the method comprising:
loading a mixture of adapter ligated, fragmented nucleic acid polymers onto a nano-deterministic lateral displacement (nanoDLD) array, the mixture comprising a first adapter ligated, fragmented nucleic acid polymer, a second adapter, ligated fragmented nucleic acid polymer, and a contaminant; and
flowing the mixture through the nanoDLD array to separate the first adapter ligated, fragmented nucleic acid polymer from the second adapter ligated, fragmented nucleic acid polymer, and each of the first and second adapter ligated, fragmented nucleic acid polymers from the contaminant;
wherein a concentration of the mixture is increased after flowing through the nanoDLD array.

16. The method of claim 15 further comprising sequencing the first and second adapter ligated fragmented nucleic acid polymers subsequent to flowing through the nanoDLD array.

17. The method of claim 15, wherein the contaminant comprises an enzyme used to fragment nucleic acid polymers.

18. The method of claim 15, wherein the mixture comprises fragmented deoxyribonucleic acid (DNA) polymers.

19. The method of claim 15, wherein loading the mixture onto the nanoDLD array comprises using a full width configuration.

20. The method of claim 15, wherein the contaminant comprises an adapter dimer.
